(19) United States
(12) Patent Application Publication
(10) Pub. No.: US 2004/0018174 A1
(43) Pub. Date: Jan. 29, 2004

(54) Cell therapy for regeneration

(75) Inventor: Maria Palasis, Wellesley, MA (US)

Correspondence Address: HALE AND DORR LLP, 300 PARK AVENUE, NEW YORK, NY, 10022, US

(73) Assignee: Boston Scientific Corporation, Natick, MA

(21) Appl. No.: 10/623,205

(22) Filed: Jul. 18, 2003

Related U.S. Application Data
(60) Provisional application No. 60/397,911, filed on Jul. 23, 2002.

(51) Int. Cl. A61K 45/00; A61K 38/18; A61K 38/19; A61K 38/20

(52) U.S. Cl. 424/93.7; 514/12; 424/85.1; 424/85.2; 435/372

(57) ABSTRACT

This invention relates to methods of autologous transplantation for the treatment of damaged or diseased tissues, muscle, or organs. This invention also relates to methods of allogeneic transplantation for the treatment of damaged tissues and organs. In the methods of the invention, cells are obtained by apheresis and implanted into the tissue, muscle, or organ to ameliorate damage or disease.

This application claims the benefit of provisional application U.S. Ser. No. 60/397,911 filed Jul. 23, 2002, which is hereby incorporated by reference.

### FIELD OF THE INVENTION

This invention relates to the field of medical treatment of injured and/or diseased organs and tissues including cardiovascular tissue. More specifically, this invention relates to the use of peripheral blood stem cells to treat damaged and diseased tissue by cellular implantation.

### BACKGROUND OF THE INVENTION

Many tissues in the body fail to regenerate independently after injury, infection, or other environmental stresses, and intervention may be required to restore function to those tissues. A notable example is the heart, whose intrinsic repair mechanisms are often inadequate to restore function after a myocardial infarction (Thurmond et al. (2001) J. Mol. Cell. Cardiol. 33:883-85). Cardiomyocytes destroyed by necrosis or apoptosis are not effectively replaced. The remaining cardiomyocytes are unable to reconstitute tissue lost to necrosis, and the heart deteriorates over time (Orlic et al. (2001) Nature, 410:701-705).

Recent attempts to ameliorate the damage caused by myocardial infarction or other disease processes have been directed to regenerating myocardial tissue by implanting a variety of stem and progenitor cells that can differentiate into cardiac muscle. Field (U.S. Pat. No. 5,602,301) has shown that the myocardium can accept grafts of skeletal myoblasts and cardiomyocytes. Taylor et al. (Nat. Med. 4:929-933, 1999) demonstrated that rabbits receiving autologous transplants of skeletal myoblasts into the myocardium after cryogenic injury showed improved myocardial performance. Mickle et al. (U.S. Pat. No. 6,110,459) showed that adult-derived cardiomyocytes, smooth muscle cells, and fibroblasts can be successfully transplanted into myocardial scar tissue. In addition, progenitor bone marrow cells have been shown to have the capacity to differentiate into myocardial muscle (Springer et al. (2001) J. Clin. Inv. 107:1355-1356). Orlic et al. (Nature, 410: 701-705, 2001) teach that adult-derived bone marrow cells can regenerate infarcted myocardium. Petersen et al. (Science, 284: 1168-1170, 1999) demonstrate that cells derived from bone marrow can also differentiate into ductular cells and hepatocytes when implanted in liver. Thus, cells derived from bone marrow possess the ability to differentiate into a variety of non-hematopoietic cell types.

Despite the desirability of obtaining autologous tissue whenever possible to avoid rejection of the implanted cells by the host immune system, stem and progenitor cell populations in skeletal muscle and cardiac muscle are not sufficiently accessible in a subject's own tissue to be readily adapted to clinical therapy (Jackson et al. (2001) J. Clin. v. 107:1395-1401). Furthermore, methods of obtaining stem cells and progenitor cells from muscle and bone marrow require invasive procedures that carry the additional risks associated with general and local anesthesia.

While the transplantation of stem cells clearly shows promise for repopulating damaged or diseased tissue, less invasive and more effective methods of obtaining stem cells and progenitor cells are needed.

### SUMMARY OF THE INVENTION

Stem cells isolated from peripheral blood are competent to differentiate into a wide variety of cell types, including non-hematopoietic cells. The invention provides methods of treating damaged or diseased tissues and organs by implanting stem cells at the site of disease or damage.

In one aspects the invention provides a method for treating damaged or diseased tissues and organs via autologous or allogeneic transplantation by using an apheresis procedure to isolate peripheral blood stem cells from a donor. These cells are then implanted or administered at the site of damage or in the diseased tissue or organ of the subject undergoing treatment. The implanted stem cells proliferate and differentiate to form stable grafts at the site of damage or disease.

In conjunction with this method, an effective amount of a mobilization factor or a combination of mobilization factors can be administered to the donor prior to apheresis, thus allowing an increased yield of stem cells obtained by apheresis. Preferred mobilization factors include, but are not limited to, granulocyte colony stimulating factor (G-CSF), granulocyte-macrophage colony stimulating factor (GM-CSF), interleukin 1 (IL-1), interleukin 3 (IL-3), stem cell factor (SCF, also known as steel factor or kit ligand), vascular endothelial growth factor (VEGF), Flt-3 ligand, platelet-derived growth factor (PDGF), epidermal growth factor (EGF), fibroblast growth factor-1 (FGF-1), fibroblast growth factor-2 (FGF-2), insulin-like growth factor-1 (IGF-1), megakaryocyte growth and development factor (MGDF), nerve growth factor (NGF), and 3-hydroxy-3-methyl glutaryl coenzyme A (HMG CoA)reductase inhibitors, and fragments and variants thereof which retain the same biological activity.

To promote the proliferation and/or differentiation of the implanted cells, an engraftment factor or a combination of engraftment factors can be administered to the subject simultaneous with or following implantation of the isolated peripheral blood stem cells, thus promoting cell proliferation, differentiation, attachment, or survival of the implanted cells. Preferred engraftment factors include, but are not limited to G-CSF, GM-CSF, IL-1, IL-3-, SCF, VEGF, Flt-3 ligand, heme oxygenase, cell survival proteins such as the cell survival kinase AKT (also known as protein kinase B), and fragments and variants thereof which retain the same biological activity, and other agents such as nitric oxide and 5-azacytidine. Other factors that stimulate or facilitate engraftment can also be administered or locally implanted, including matrices for cell attachment, such as extracellular matrix, and natural polymers such as fibronectin, laminin, and collagen.

In another aspect, the invention is directed to a method of treating striated muscle tissue that has suffered damage or disease. Again, apheresis is used to isolate stem cells from the peripheral blood. The isolated cells are then implanted at the desired site of damage or disease. The implanted peripheral blood stem cells proliferate and differentiate into striated muscle cells, and form stable grafts at the site of damage or disease. By forming stable grafts, the implanted cells can repopulate lost tissue or provide healthy tissue at sites of scarring or necrosis.

In some embodiments of this aspect of the invention, an effective amount of a mobilization factor or a combination of mobilization factors is administered to the donor prior to performing apheresis, as described above. In addition, or alternatively, an effective amount of an engraftment factor or a combination of engraftment factors can be administered to the subject at the same time as or following implantation of the stem cells.

In yet another aspect, the invention provides a minimally invasive method of treating an ischemic or damaged organ. Apheresis is used to isolate stem cells from peripheral blood, as described above. The isolated cells are implanted in the ischemic organ at the site of damage or disease and then proliferate and differentiate to form stable grafts. In a preferred embodiment, the damaged or diseased organ treated by the method of the invention is heart. In another preferred embodiment, the damaged-or diseased organ treated by the method of the invention is liver. In another preferred embodiment, the damaged or diseased organ treated by the method the invention is brain.

As described above, in some embodiments of this aspect of the invention, an effective amount of a mobilization factor or a combination of mobilization factors is administered to the donor prior to performing apheresis. Alternatively or in addition, an effective amount of an engraftment factor or a combination of engraftment factors is administered to the subject concurrently with or following implantation of the isolated stem cells.

### DETAILED DESCRIPTION OF THE INVENTION

The issued U.S. patents, published and allowed applications, and references cited herein are hereby incorporated by reference.

The present invention relates to methods of autologous transplantation using stem cells isolated from peripheral blood. Autologous transplantation is the transplantation of tissues or cells from a subject's own body rather than from a donor individual. The, present invention also relates to methods of allogeneic transplantation. Allogeneic transplantation is the transplantation of tissues or cells from a genetically non-identical individual of the same species. These methods are useful for treating tissues and organs. In accordance with the invention, peripheral blood stem cells isolated by apheresis are introduced to damaged or diseased organs, muscle, or tissues to ameliorate the damage or disease therein.

The tissue or organ to be treated by the method of the present invention has been damaged or is diseased, and such damage or disease may have occurred in a variety of ways. For example, damage or disease may result from ischemia brought on by an infarction, mechanical injury, as a consequence of surgery, or as a result of an inherited (genetic) condition. Damage can also result from chemotherapy or irradiation. Alternatively, the damage or disease can result from a bacterial or viral infection, or the subject may suffer from a degenerative disease that causes progressive loss of healthy cells.

As used herein, the term “damaged or diseased tissue” means tissue in which cells have been lost or have died due to insufficient blood supply, mechanical injury, infection, irradiation, trauma, disease, or other insult. For example, damaged and or diseased tissue includes, but is not limited to, scar tissue, and tissue that is torn, crushed, or has undergone necrosis resulting from blood loss. By “necrosis” is meant pathologic cell death following irreversible damage to the cell. The damaged or diseased tissue can be distinguished from the surrounding tissue, for example, by physical inconsistency or discontinuity.

A subject to be treated according to the method of the invention is one who has suffered an injury or has an illness or disorder that results in tissue damage. In certain cases, the injury is an infarction that results in tissue necrosis, and more particularly, a myocardial infarction. Such subjects include humans and animals, such as laboratory animals or feed animals, including, but not limited to, mice, rats, rabbits, dogs, cats, cattle, swine, non-human primates, and others. Preferably, the subject is a human.

The methods of the invention can be used to treat any organs and tissues in need thereof. Examples of tissues include, but are not limited to, bone, cartilage, and striated muscle, which includes cardiac muscle and skeletal muscle. Non-limiting examples of organs that can be treated by the methods of the invention include heart, liver, brain, kidney, intestine, lung, eye, pancreas, bladder, and spinal cord.

A variety of types of damage and disease can be treated. For example, ischemia can result in tissue necrosis. A degenerative disease can result in loss of cells to apoptosis and necrosis. Tissue can be torn, crushed, scarred, weakened, or lost as a result of a mechanical injury. Scar tissue may have formed at the site of damage or disease caused by any of the injuries and illnesses listed above. An organ may be reduced in size due to loss of healthy cells as a result of ischemia or other disease. Other types of damage and disease can be treated as well. Such damage and disease can be ameliorated by repopulation or augmentation of the affected area by healthy cells of the appropriate cell type, as provided by the invention.

Repopulation and/or augmentation of the affected area is accomplished by introducing stem cells to the site of damage or disease. It has been shown that stem cells obtained from peripheral blood can reconstitute the hematopoietic tissues of subjects undergoing cytoreductive therapy such as chemotherapy or radiation therapy (Cutler and Antin (2000) Stem Cells 19:108-117; Champlin et al. (2000) Blood 95: 3702-3709). In the present methods, autologous transplantation of stem cells isolated from peripheral blood is used to repopulate or augment non-hematopoietic tissues and organs. The use of autologous cells eliminates the risk of rejection of the implanted tissue by the recipient, and the use of cells harvested from peripheral blood provides a less invasive method of obtaining stem cells than obtaining cells from bone marrow, skeletal muscle, or other internal organs or tissues.

Alternatively, allogeneic transplantation of stem cells isolated from the peripheral blood of a donor individual are used to repopulate or augment non-hematopoietic tissues and organs. When allogeneic cells are used, it is preferred that the donor and the subject be HLA-compatible to the extent possible. When allogeneic cells are used, methods of suppressing the immune system of the recipient can be used, including, but not limited to, the administration of immunosuppressive drugs, radiation, chemotherapeutics, or antibody masking techniques or agents.

Stem cells are distinguished from differentiated cells, the latter being defined as those cells that may or may not have the capacity to proliferate, i.e., self-replicate, but that are unable to undergo further differentiation to a different cell type under normal physiological conditions. As used herein, stem cells include all the cells in a lineage of differentiation and proliferation prior to the most differentiated or fully mature cells. Thus, stem cells include totipotent cells, pluripotent cells, and progenitor cells. Stem cells include, for example, hematopoietic stem cells, which give rise to a wide variety of different cell types in the lymphoid, myeloid, and erythroid lineages. Stem cells also include, for example, the skin progenitor, which is capable of differentiation to only one type of cell, but which is not itself fully mature or differentiated, and is thus included in the definition of a stem cell.

Stem cells can be distinguished by their ability to form mature cell types in cell culture or by the presence or absence of certain cell surface markers. For example, certain subpopulations of stem cells express the surface antigens c-kit and Thy-1, but are lineage-negative and CD38-negative. Stem cells according to the invention include various subpopulations of cells. Cells known as mesenchymal stem cells, hematopoietic stem cells, and side population cells are representative stem cells useful in the present invention. Accordingly, stem cells may express cell surface antigens characteristic of these subpopulations of stem cells. Stem cells according to the invention also include mixed populations of cells comprising different subpopulations of cells, such as mixtures of hematopoietic stem cells and mesenchymal stem cells.

Peripheral blood is defined as blood that is found anywhere in the circulatory system, including the heart, arteries, veins, and capillaries.

Methods of obtaining stem cells from peripheral blood by apheresis are known in the art (see, e.g., Moog and Muller (2001) Ann. Hematol. 77:143-147). Apheresis is used to isolate cellular components from the blood and involves removing blood from a subject, subjecting the blood to a separation method to remove certain components, and reinfusing the blood into the subject. By varying the separation method, apheresis can be adapted to isolate different cellular components from the blood. For example, some separation methods isolate cellular fractions with high concentrations of platelets, other methods isolate cellular fractions with high concentrations of erythrocytes, and others isolate cellular fractions with high concentrations of mononuclear cells.

In the methods of the invention, apheresis is used to isolate stem cells from the blood. These cells are found in cellular fractions with high numbers of mononuclear cells, fractions that are also known as the “buffy layer” or “buffy coat.” Thus, the separation method in the apheresis step isolates these mononuclear cell fractions. Other apheresis protocols, such as those used to collect platelets or erythrocytes, are not used. In methods where multiple cellular fractions are obtained, the mononuclear cell fraction is used, and not, for example, platelet or erythrocyte fractions.

Access to peripheral blood is preferably gained through a peripheral vein, though in some cases a central line can be used. Apheresis can be performed using manually operated or fully automated devices for blood separation, such as the Haemonetics (Braintree, Mass.) V50 blood separator or the Baxter CS 3000(Baxter, Deerfield Ill.), the Fresenius AS 104 and the Fresenius AS TEC 104 (Fresenius, Bad Homburg, Germany), the Excel (Dideco, Mirandola, Italy), and other devices.

An additional step to enrich cell fractions obtained by apheresis containing stem cells and to remove unwanted cellular components can also be performed. For example, the cellular fractions obtained by apheresis can be further separated by density gradient centrifugation to enrich for mononuclear cell populations that include stem cells (see e.g. Current Protocols in Immunology, ed. John E. Coligan, John Wiley & Sons, Inc., 1993). Stem cell fractions can also be further enriched or purified by employing any useful separation methods, such as, but not limited to, those based on cell surface antigens characteristically expressed by certain types of stem cells. For example, fluorescence-activated cell sorting can be used to isolate stem cells expressing c-kit or to remove lineage-positive differentiated cells. Alternatively, cells can be sorted by mixing with magnetic beads coated with monoclonal antibodies against a cell surface antigen characteristically expressed by stem cells.

Accordingly, the stem cells administered to the patient can vary in composition depending on the method of isolation and further purification employed. For example, the stem cells can include a mixed population of different subpopulations of stem cells, or stem cells at different stages of progression in a cellular lineage. Alternatively, separation methods employed subsequent to apheresis can enrich for stem cells or particular subpopulations therein.

The isolated stem cells can be used the same day or cryogenically stored for later use. Cryogenic preservation methods are known in the art. The stem cells can also be expanded ex vivo using known methods (see, e.g., Ziegler and Kanz (1998) Current Opinion in Hematology 5:434-440 or U.S. Pat. No. 5,541,103). The cells can also be subjected to other manipulations including the introduction of exogenous nucleic acids. Methods for introducing nucleic acids to mammalian cells are known in the art and include, but are not limited to, transfection, electroporation, lipofection, and other methods. Nucleic acids can be introduced prior to or following expansion of the cells.

The isolated stem cells are administered to the subject, or delivered to the specific organ or tissue, using a number of known methods. For example, the cells can be delivered to the tissue by intramuscular or intramyocardial injection using a needle or other delivery device. Alternatively, the cells can be delivered by a catheter, such as a Stilleto catheter (Boston Scientific, Natick Mass.). The cells can also be delivered using surgical procedures, or during surgical procedures if appropriate; or they can be delivered by intracoronary infusion, intraarterial infusion, intravenous infusion, or retrograde perfusion. While non-surgical methods are preferred when possible, the route and method of introduction can vary depending on the tissue to be treated as well as the size of the damaged or diseased area. The cells can be delivered in a single procedure, or in more than one procedure. The number of cells delivered to the site of damage or disease can vary depending on the size of the damaged or diseased area and the severity of damage or disease progression, but can range from 1×10⁴−1×10⁸ cells, and preferably ranges from 1×10⁵−1×10⁷ cells.

In the methods described herein, the cells can be included in formulations suitable for administration into the bloodstream or for administration directly into tissues or organs. A suitable format is determined by a medical practitioner for each patient, tissue, and organ, according to standard procedures. Suitable pharmaceutically acceptable carriers and their formulation are known in the art (see, e.g., Remington's Pharmaceutical Sciences 16^{th} edition, Osol, A. Ed. 1980) Cells of the present invention are preferably formulated in solution at a pH from about 6.5 to about 8.5. Excipients to bring the solution to isotonicity can also be added, for example, 4.5% mannitol or 0.9% sodium chloride, pH buffered with art-known buffer solutions, such as sodium phosphate. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes.

To obtain optimal yields of stem cells from peripheral blood, compounds that enhance mobilization of stem cells can be administered to the donor (or to the subject donor when using autologous cells)prior to apheresis. By “mobilization” is meant an increase in the population of stem cells or specific subpopulations of cells in peripheral blood. As used herein, “mobilization factors” are proteins or other chemical agents that, when administered to a donor increase the number of stem cells present in the peripheral blood of the donor. Thus, in some embodiments of the invention, an effective amount of a mobilization factor or a combination of mobilization factors is administered to the donor prior to performing apheresis.

As used herein, an “effective amount” of a mobilization factor refers to that amount of a mobilization factor necessary to administer to a subject to promote mobilization of stem cells into the periphery. Such amounts depend on the mobilization factor and its bioavailability, but can range from 0.1-1000 μg/kg of body weight. Effective amounts can be readily determined by one of ordinary skill in the art.

Preferred mobilization factors according to the invention include, but are not limited to, G-CSF, GM-CSF, IL-1, IL-3, SCF, Flt-3 ligand, VEGF, PDGF, EGF, FGF-1, FGF-2, IGF-1, MGDF, NGF, inhibitors of HMG CoA reductase, and bioactive or functional variants of these proteins, including fusion proteins and chimeric proteins. Many mobilization factors can be obtained commercially from Amgen (Thousand Oaks, Calif.), or Immunex (Seattle, Wash.). Alternatively, the mobilization factors can be synthesized by recombinant or other biosynthetic means and purified by methods known in the art.

The mobilization factors can be administered in any effective manner, including intravenously, intramuscularly, or subcutaneously (see, e.g. U.S. Pat. No. 5,199,942; U.S. Pat. No. 5,032,395). The mobilization factor can be administered in a single dose prior to apheresis or in repeated doses administered over several days prior to apheresis. For example, the donor can receive from 0.1-1000 μg/kg of body weight of the mobilization factor every 12 or 24 hours for five days prior to apheresis. Alternatively, the donor can receive 0.1-1000 μg/kg of body weight of the mobilization factor for a single day prior to apheresis. The schedule of administration varies depending on the particular mobilization factor or combination of mobilization factors to be used and can be determined by those of skill in the art. For example, SCF is preferably administered at 10 μg/kg to 800 μg/kg of body weight, GM-CSF is preferably administered at from 1-100 μg/kg of body weight, and more preferably from 1-50 μg/kg of body weight. Preferred dosage ranges for other growth factors can be determined by those of skill in the art. Preferably, the mobilization factor or factors are administered to the patent for from one to ten days prior to apheresis.

Alternatively, or in addition, the method of the invention also includes the administration of compounds to promote engraftment of the implanted stem cells. By “engraftment” is meant that the implanted cells proliferate and/or differentiate to repopulate the tissue or augment the cells at the site of damage or disease. “Engraftment factors” are defined as proteins or other chemicals that stimulate or induce the implanted stem cells to elicit specific responses from those cells, such as cell migration, proliferation, differentiation, and cell adhesion, cell attachment, and cell survival. Representative proteins include growth factors, which are administered to the subject after implantation of the stem cells (see, e.g., Qu et al. (1998) J. Cell Biol. 142:1257-1267; U.S. Pat. No. 05,199,942; U.S. Pat. No. 5,602,301).

The subject can be treated with an effective amount of one or more engraftment factors prior to, at the same time as, or following, implantation of the stem cells to promote the formation of stable grafts. Preferred engraftment factors include G-CSF, GM-CSF, IL-1, IL-3, SCF, VEGF, Flt-3 ligand, heme oxygenase, cell survival proteins such as the cell survival kinase AKT (also known as protein kinase B) and other agents such as nnitric oxide and 5-azacytidine. Bioactive and functional variants of these factors including fusion proteins and chimeric proteins can also be used. However, other engraftment factors, including tissue-specific engraftment factors and matrices for cell attachment, such as extracellular matrix, submucosal tissue and natural polymers such as fibronectin, laminin, and collagen can be used.

As used herein, an “effective amount” of an engraftment factor refers to that amount of a therapeutically active engraftment factor necessary to promote the proliferation and differentiation of implanted cells at the site of damage. Such amounts depend on the engraftment factor or combination of engraftment factors, but can range from 0.1-1000 μg/kg of body weight. Effective amounts can readily be determined by one of skill in the art.

The factors for promoting engraftment can be administered in any suitable and efficacious way, including orally, intravenously, intramuscularly, subcutaneously or surgical implantation. The schedule of administration varies depending on the particular engraftment factor or combination of engraftment factors to be used, and the tissue or organ into which the cells are implanted. For example, the engraftment factor can be delivered with the cells by injection. The engraftment factor can also be delivered to the organ prior to administration of the cells. The factor can also be delivered separately by intravenous, intracoronary, or oral administration. The engraftment factor can be delivered in a controlled release form. The engraftment factor can also be delivered over several days after the cells are administered. The engraftment factor can be delivered any number of days after administration of the cells, preferably within 28 days of administration of the cells, and more preferably within 14 days after administration of the cells.

The mobilization factors and engraftment factors administered in the methods of the invention can also be prepared as pharmaceutical formulations. Pharmaceutical formulations of the mobilization factors and engraftment factors of the invention are prepared by mixing those entities having the desired degree of purity with optional physiologically acceptable carriers, excipients, or stabilizers, in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride; phenol, butyl, or benzyl alcohol; alkyl parabens such as methyl or propyl paraben; catechol; resorcinol; cyclohexinol; 3-pentanol; and me-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as. EDTA; sugars such as sucrose, mannitol, trehalose, or sorbitol; salt-forming counter-ions such as sodium; metal complexes (e.g., Zn-protein complexes); and/or non-ionic surfactants such as TWEEN™, PLURONICS™, or polyethylene glycol (PEG).

The present invention also provides a method for autologous or allogenic transplantation of stem cells to treat damaged or diseased striated muscle tissue, such as cardiac muscle or skeletal muscle. As used herein, “striated muscle” is meant to encompass muscle tissue that exhibits regularly spaced transverse bands along the length of the muscle fiber. Striated muscle tissue includes both cardiac muscle and skeletal muscle. For autologus transplantation, stem cells are isolated by apheresis from the peripheral blood of the subject to be treated in accordance with the present invention and are implanted at the site of damaged or diseased striated muscle tissue. For allogenic transplantation the stem cells are isolated from the appropriate donor and implanted as above.

The subject to be treated according to the method of the invention has suffered damage or disease to cardiac muscle, or to skeletal muscle. For example, the method is useful for a subject who experienced a myocardial infarction. Insufficient blood supply to cardiac or skeletal muscle can in turn lead to tissue necrosis, the formation of scar tissue, or the thinning or weakening of cardiac or skeletal muscle. The method of the invention is also useful for a subject who experienced a mechanical injury, such as that caused by an accident or other external trauma that resulted in damage or disease in muscle tissue. Alternatively, the method can be used for subjects suffering from a disease such as, but not limited to, muscular dystrophy that causes progressive loss of healthy striated muscle cells. Such damage is ameliorated, for example, by regenerating and/or repopulating the affected area with stem cells able to differentiate into striated muscle cells.

As described above, the stem cells used according to the method of the invention are stem cells obtained from peripheral blood. These cells are capable of proliferating and differentiating into multiple muscle cell types, such as cardiomyocytes or skeletal myocytes. Stem cells for repopulating skeletal and cardiac muscle can be distinguished by their ability to form striated muscle cells in cell culture assays or by characteristic expression patterns of cell surface antigens, such as, but not limited to, expression of antigens reactive with SH2, SH3, and SH4 monoclonal antibodies (see U.S. Pat. No. 5,486,359).

Stem cells for repopulating striated muscle are obtained by apheresis and stored or expanded as described herein above.

The isolated stem cells can be delivered to the muscle using any number of methods. Preferably, the cells are injected at the damaged or diseased site. Delivery to myocardium can be accomplished using a catheter, needle, needle-free injector, balloon catheter, channeling device, patch, or other appropriate medical device for introduction into the myocardium. Alternatively, a surgical approach may be necessary for delivery to myocardium, either via open chest or via thoracoscopy. In the case of skeletal muscle, percutaneous or intramuscular injection can be used to deliver the cells directly to the affected tissue, as can channeling devices or other medical devices or methods. Cells can also be delivered to cardiac or skeletal muscle by needle, needle-free injector, access through the coronary or venous system, or retrograde perfusion.

As described above, compounds to promote the mobilization of stem cells prior to collection from peripheral blood can be used in the methods of the invention. Preferred mobilization factors include, but are not limited to, G-CSF, GM-CSF, IL-1, IL-3, SCF, VEGF, Flt-3 ligand, PDGF, EGF, FGF-1, FGF-2, IGF-1, MGDF, NGF, HMG CoA reductase inhibitors, and fragments and variants thereof which retain the same biological activity, and fusion proteins and chimeric proteins including these factors.

As described above, engraftment factors can be used in addition to the methods described above. Preferred engraftment factors include, but are not limited to, GM-CSF, G-CSF, IL-1, IL-3, SCF, VEGF, Flt-3 ligand, heme oxyganese, cell survival factors, such as AKT, nitric oxide, 5-azacytidine and mobilization/attachment factors such as collagen, laminin, fibronectin, and fragments and variants thereof which retain the same biological activity, and fusion proteins and chimeric proteins including these factors.

In another aspect, the invention provides a method of treating a subject having an ischemic organ. An “ischemic organ” according to the invention is one that suffers from an insufficient supply of blood to the organ. Ischemia can be caused, for example, by a mechanical obstruction such as arterial narrowing or the presence of scar tissue following injury. Organs that can be treated by the method of the invention include, but are not limited to, heart, brain, liver, kidney, bone, lung, intestine, spleen, eye, and bladder. Any organ or tissue that has suffered damage or disease as a result of ischemia can also be treated using the method of the present invention.

A subject treated by the method of the invention has experienced damage or disease to an organ or tissue, for example, caused by an insufficient blood supply to that organ or tissue. Ischemia can be caused by, for example, an infarct, such as an infarct of the brain, spleen, kidney, intestine, or lung. Ischemia may also develop as a consequence of surgery, such as resection of the liver. A mechanical injury that results in loss of blood to an organ or tissue, such as an accident or other external trauma can also cause ischemia.

As described above, for this aspect of the invention, the stem cells administered to the subject and delivered to the organ are isolated from the subject's peripheral blood by apheresis. As described herein, the isolated cells can be used immediately, stored for later use, expanded ex vivo, recombinantly manipulated, or any combination thereof.

A number of methods can be employed to deliver the stem cells to the particular organ or to be treated. For example, the cells can be delivered by intravenous or intraarterial injection to the organ to be treated. When possible, delivery by injection through a syringe, catheter, or similar medical device is preferred. However, the method and route of introduction of cells varies depending on the tissue to be treated as well as other factors such as the size of the area to be treated and the route for gaining access to the damaged or diseased area. Those of skill in the art can readily determine the appropriate method for introducing the cells to the damaged or diseased area. For example, cells can be delivered to the liver by direct injection or infusion through hepatic artery. Cells can be delivered to the brain by microneedle injection or by infusion into the cerebrovascular space.

As described previously, the subject can be treated according to this aspect of the invention with mobilization factors to mobilize stem cells prior to their isolation from peripheral blood. Preferred mobilization factors include GM-CSF, G-CSF, IL-1, IL-3, SCF, VEGF, Flt-3 ligand, PDGF, EGF, FGF-1, FGF-2, IGF-1, MGDF, NGF, HMG CoA reductase inhibitors, and fragments and variants thereof which retain the same biological activity, including fusion proteins and chimeric proteins.

Alternatively, or in addition, the subject can also be treated with engraftment factors following implantation of the stem cells in order to promote the proliferation and differentiation of the implanted cells such that the damaged or diseased organ or tissue is repopulated with new cells. Preferred engraftment factors according to the invention include GM-CSF, G-CSF, IL-1, IL-3, SCF, VEGF, Flt-3 ligand, heme oxygenase, cell survival factors, and attachment factors such as collagen, laminin, fibronectin, and fragments and variants thereof which retain the same biological activity including fusion proteins and chimeric proteins. Other agents, such as nitric oxide and 5-azacytidine can also be used to promote engraftment.

The engraftment factor or combination of engraftment factors can be administered at the same time as the implanted cells or after implantation of the cells. The amount and schedule of administration of engraftment factors can vary depending on the particular engraftment factor or combination of engraftment factors to be used.

It is to be understood and expected that variations in the principles of the invention herein disclosed in an exemplary embodiment may be made by one skilled in the art and it is intended that such modifications, changes, and substitutions are to be included within the scope of the present invention.

### EXAMPLES

### Example 1

### Mouse Model of Hindlimb Ischemia

Peripheral blood cells are obtained from human donors by apheresis. In one group of donors, total mononuclear cells are collected by density gradient centrifugation. In another group, CD34+ cells are collected by density gradient centrifugation followed by fluorescence-activated cell sorting. The collected cells are labeled with the fluorescent dye, Dil, prior to introduction into mice to permit detection of engrafted cells.

For administration of human stem peripheral blood cells obtained by apheresis, C57BL/6Jx129/SV background athymic nude mice are used to avoid potential graft-versus host complications. To induce ischemia, animals are anesthetized intraperitoneally with pentobarbital. The proximal end of one femoral artery and the distal portion of the corresponding saphenous artery are ligated. The artery and all side branches are dissected free and excised. When the limb is severely ischemic, a total of approximately 5×10⁵ human mononuclear or CD34⁺ cells are injected directly into the ischemic hindlimb at five to ten locations. Histologic sections of limbs are examined 1, 2, 4, and 6 weeks later for the presence of Dil-labeled human cells in the ischemic hindlimb.

### Example 2

### Large Animal Model

Juvenile crossbred pigs weighing approximately 35 to 40 kg undergo left lateral thoracotomy. An ameroid constrictor is placed around the proximal LCX just distal to the main stem of the left coronary artery using an ameroid constrictor matching the size of the vessel, typically 1.75, 2.00, or 2.25 inner diameter. Baseline measurements of cardiac function are obtained four weeks after placement of the ameroid constrictor. The measurements include coronary angiography, dobutamine stress echocardiography, and blood flow measurements by injection of microspheres at rest and at atrial pacing of 180 beats per minute (bpm).

Stem cells are isolated from the peripheral blood of anesthetized pigs by apheresis. Approximately 2×10⁶ cells/kg of body weight are collected from each pig.

Pigs are divided into treatment groups: an experimental group receives 15 injections of peripheral blood stem cells in 100 μL and a control group receives injections of the same volume of heparinized saline. Animals are randomized to injections of peripheral blood cells or heparanized saline. Injections are performed using a stiletto injection catheter. Injections are evenly distributed about 1 cm apart in the ischemic region and its boundaries and the non-ischemic territory. Injection sites are marked using fluorescent microspheres. An additional control group of animals without myocardial ischemia is studied.

Four weeks after the injections, i.e., eight weeks after implantation of the ameroid constrictor, the baseline measurements are repeated. The animals are sacrificed and the tissue samples are retained for histological analysis.

### Example 3

### Human Patients

Patients are selected who have suffered myocardial infarction resulting in scarring of the myocardium. G-CSF is administered subcutaneously at 2-24 μg/kg body weight daily for three days prior to apheresis. Apheresis is performed and 2×10⁶ cells/kg of body weight are collected. The collected cells are cryogenically stored for later use.

Cells are delivered to the myocardium by Stilletto injection catheter (Boston Scientific, Natick Mass.). Cardiac function is monitored by PET imaging and by treadmill testing at three months, six months, and twelve months following treatment.

Those skilled in the art will recognize, or be able to ascertain, using no more than routine experimentation, numerous equivalents to the specific embodiments described herein. Such equivalents are considered to be within the scope of this invention, and are covered by the following claims.

What is claimed is:

1. A method of treating damaged or diseased tissue of a subject, comprising:
(a) isolating stem cells from peripheral blood of a donor by apheresis; and
(b) implanting a population of the isolated stem cells into tissue in need of treatment, whereby implantation of the stem cells ameliorates damage or disease of the tissue.

2. The method of claim 1, wherein the donor is the subject to be treated.

3. The method of claim 1, wherein the stem cells are obtained from the peripheral blood of a donor who is HLA-matched to the subject to be treated.

4. The method of claim 1, wherein the subject is a human.

5. The method of claim 1, 2 or 3, which further comprises administering an effective amount of at least one mobilization factor to the donor, prior to step (a), in an amount and for a time sufficient to increase the peripheral blood stem cells in the donor.

6. The method of claim 5, wherein the mobilization factor is selected from the group consisting of G-CSF, GM-CSF, IL-1, IL-3, SCF, Flt-3 ligand, VEGF, PDGF, EGF, FGF-1, FGF-2, IGF-1, MGDF, NGF, and HMG CoA reductase inhibitors.

7. The method of claim 1, which further comprises administering an engraftment factor concurrently with or following the implanting step in an amount and for a time sufficient to promote the engraftment of the cells in the subject.

8. The method of claim 7, wherein the engraftment factor is selected from the group consisting of GM-CSF, G-CSF, Il-1, IL-3, SCF, VEGF, Flt-3 ligand, Akt, hemeoxygenase, nitric oxide, 5-azacytidine, collagen, laminin, and fibronectin.

9. The method of claim 1, wherein the stem cells are expanded ex vivo prior to step (b).

10. The method of claim 1, which further comprises the step of fractionating the cells prior to implantation.

11. The method of claim 10, wherein the cells are fractionated by fluorescence-activated cell sorting.

12. The method of claim 10, wherein the cells are fractionated by density gradient centrifugation.

13. The method of claim 1, wherein the cells are implanted at the site of disease or damage.

14. A method of treating damaged or diseased striated muscle tissue of a subject, which comprises:
(a) isolating stem cells from peripheral blood of a donor by apheresis; and
(b) implanting a population of the isolated stem cells into said striated muscle tissue in need of treatment,
whereby implantation of the stem cells ameliorates damage or disease of the striated muscle tissue.

15. The method of claim 14, wherein the donor is the subject to be treated.

16. The method of claim 14, wherein the stem cells are obtained from the peripheral blood of a donor who is HLA-matched to the subject to be treated.

17. The method of claim 14, wherein the muscle tissue is ischemic.

18. The method of claim 14, wherein the muscle tissue is necrotic.

19. The method of claim 14, wherein the subject is a human.

20. The method of claim 14, wherein the striated muscle is myocardium.

21. The method of claim 14, wherein the striated muscle is skeletal muscle.

22. The method of claim 14, 15 or 16, which further comprises administering at least one mobilization factor to the donor from whom stem cells are obtained, prior to step (a), in an amount and for a time sufficient to increase the peripheral blood stem cells in the subject.

23. The method of claim 22, wherein the mobilization factor is selected from the group consisting of G-CSF, GM-CSF, IL-1, IL-3, SCF, Flt-3 ligand, VEGF, PDGF, EGF, FGF-1, FGF-2, IGF-1, MGDF, NGF, and HMG CoA reductase inhibitors.

24. The method of claim 14, wherein an effective amount of at least one engraftment factor is administered to the subject concurrently with or following the implanting step in an amount and for a time sufficient to promote engraftment of the implanted cells in the subject.

25. The method of claim 24, wherein the engraftment factor is selected from the group consisting of GM-CSF, G-CSF, IL-1, IL-3, SCF, VEGF, Flt-3 ligand, Akt, hemeoxygenase, nitric oxide, 5-azacytidine, collagen, laminin, and fibronectin.

26. The method of claim 14, wherein the stem cells are expanded ex vivo prior to step (b).

27. The method of claim 14, which further comprises the step of fractionating the cells prior to implantation.

28. The method of claim 27, wherein the cells are fractionated by fluorescence-activated cell sorting.

29. The method of claim 27, wherein the cells are fractionated by density gradient centrifugation.

30. The method of claim 14, wherein the cells are implanted at the site of disease or damage.

31. A method of treating an ischemic organ in a subject, which comprises:
(a) isolating stem cells from peripheral blood of a donor by apheresis; and
(b) implanting the isolated stem cells into the ischemic organ,
whereby implantation of the stem cells ameliorates the damage or disease in the ischemic organ.

32. The method of claim 31, wherein the donor is the subject to be treated.

33. The method of claim 31, wherein the stem cells are obtained from the peripheral blood of a donor who is HLA-matched to the subject to be treated.

34. The method of claim 31, wherein the subject is a human.

35. The method of claim 31, wherein the damaged or diseased organ is heart.

36. The method of claim 31, wherein the organ is liver.

37. The method of claim 31, 32, or 33, which further comprises administering at least one mobilization factor to the donor from whom the stem cells are obtained, prior to step (a), in an amount and for a time sufficient to increase the peripheral blood stem cells in the subject.

38. The method of claim 37, wherein the mobilization factor is selected from the group consisting of G-CSF, GM-CSF, IL-1, IL-3, SCF, Flt-3 ligand, VEGF, PDGF, EGF, FGF-1, FGF-2, IGF-1, MGDF, NGF, and HMG CoA reductase inhibitors.

39. The method of claim 31, which further comprises administering at least one engraftment factor to the subject concurrently with or following the implanting step in an amount and for a time sufficient to promote the engraftment of the cells in the subject.

40. The method of claim 39, wherein the engraftment factor is selected from the group consisting of GM-CSF, G-CSF, IL-1, IL-3, SCF, VEGF, Flt-3 ligand, Akt, hemeoxygenase, nitric oxide, 5-azacytidine, collagen, laminin, and fibronectin.

41. The method of claim 31, wherein the stem cells are expanded ex vivo prior to step. (b).

42. The method of claim 31, which further comprises the step of fractionating the cells prior to implantation.

43. The method of claim 42, wherein the cells are fractionated by fluorescence-activated cell sorting.

44. The method of claim 42, wherein the cells are fractionated by density gradient centrifugation.

45. The method of claim 31, wherein the cells are implanted at the site of disease or damage.
